SEQUENCE LISTING (21 sequences)

SEQ ID NO: 1 - protein, 5 aa, Artificial Sequence
  synthetic
  MOD_RES (1)..(1): 2,2,4,6,7-pentamethyldihydrobenzofurane
  MOD_RES (2)..(4): tert-Butyl
RYTNG

SEQ ID NO: 2 - protein, 5 aa, Artificial Sequence
  synthetic
  MOD_RES (1)..(1): 2,2,4,6,7-pentamethyldihydrobenzofurane
  MOD_RES (1)..(1): N-Tris(2,4,6-trimethoxypheyl)phosphonium
  MOD_RES (2)..(4): tert-Butyl
RYTNG

SEQ ID NO: 3 - protein, 5 aa, Artificial Sequence
  synthetic
  MOD_RES (1)..(1): N-Tris(2,4,6-trimethoxypheyl)phosphonium
RYTNG

SEQ ID NO: 4 - protein, 8 aa, Artificial Sequence
  synthetic
  MOD_RES (2)..(2): toluenesulfonyl
  MOD_RES (4)..(4): 2-chlorobenzyloxycarbonyl
  MOD_RES (5)..(5): benzyl ester
  MOD_RES (7)..(7): benzyl ester
LRAKSQDG

SEQ ID NO: 5 - protein, 8 aa, Artificial Sequence
  synthetic
LRAKSQDG

SEQ ID NO: 6 - protein, 8 aa, Artificial Sequence
  synthetic
  MOD_RES (1)..(1): N-Tris(2,4,6-trimethoxypheyl)phosphonium
LRAKSQDG

SEQ ID NO: 7 - protein, 6 aa, Artificial Sequence
  synthetic
  MOD_RES (1)..(1): N-Tris(2,4,6-trimethoxypheyl)phosphonium
LKSQDG

SEQ ID NO: 8 - protein, 6 aa, Artificial Sequence
  synthetic
  MOD_RES (1)..(1): N-Tris(2,4,6-trimethoxypheyl)phosphonium
LNSQDG

SEQ ID NO: 9 - protein, 6 aa, Artificial Sequence
  synthetic
  MOD_RES (1)..(1): N-Tris(2,4,6-trimethoxypheyl)phosphonium
LESQDG

SEQ ID NO: 10 - protein, 6 aa, Artificial Sequence
  synthetic
  MOD_RES (1)..(1): N-Tris(2,4,6-trimethoxypheyl)phosphonium
LTSQDG

SEQ ID NO: 11 - protein, 6 aa, Artificial Sequence
  synthetic
  MOD_RES (1)..(1): N-Tris(2,4,6-trimethoxypheyl)phosphonium
LWSQDG

SEQ ID NO: 12 - protein, 6 aa, Artificial Sequence
  synthetic
  MOD_RES (1)..(1): N-Tris(2,4,6-trimethoxypheyl)phosphonium
LHSQDG

SEQ ID NO: 13 - protein, 6 aa, Artificial Sequence
  synthetic
  MOD_RES (1)..(1): N-Tris(2,4,6-trimethoxypheyl)phosphonium
LPSQDG

SEQ ID NO: 14 - protein, 6 aa, Artificial Sequence
  synthetic
  MOD_RES (1)..(1): N-Tris(2,4,6-trimethoxypheyl)phosphonium
LRSQDG

SEQ ID NO: 15 - protein, 6 aa, Artificial Sequence
  synthetic
  MOD_RES (1)..(1): N-Tris(2,4,6-trimethoxypheyl)phosphonium
LYSQDG

SEQ ID NO: 16 - protein, 6 aa, Artificial Sequence
  synthetic
  MOD_RES (1)..(1): N-Tris(2,4,6-trimethoxypheyl)phosphonium
LISQDG

SEQ ID NO: 17 - protein, 10 aa, Artificial Sequence
  synthetic
  MOD_RES (1)..(1): N-Tris(2,4,6-trimethoxypheyl)phosphonium
LKNAKQSQDG

SEQ ID NO: 18 - protein, 4 aa, Artificial Sequence
  synthetic
  MOD_RES (1)..(1): N-Tris(2,4,6-trimethoxypheyl)phosphonium
LKNA

SEQ ID NO: 19 - protein, 6 aa, Artificial Sequence
  synthetic
  MOD_RES (1)..(1): N-Tris(2,4,6-trimethoxypheyl)phosphonium
  misc_feature (2)..(2): Xaa can be any naturally occurring amino acid
LXSQDG

SEQ ID NO: 20 - protein, 4 aa, Artificial Sequence
  synthetic
  MOD_RES (1)..(1): N-Tris(2,4,6-trimethoxypheyl)phosphonium
  misc_feature (2)..(2): Xaa can be any naturally occurring amino acid
LXSQ

SEQ ID NO: 21 - protein, 4 aa, Artificial Sequence
  synthetic
  MOD_RES (1)..(1): N-Tris(2,4,6-trimethoxypheyl)phosphonium
LHSQ

(19) United States
(12) Patent Application Publication
(10) Pub. No.: US 2019/0025324 A1
(43) Pub. Date: Jan. 24, 2019

(54) PEPTIDE SEQUENCING DIRECTLY FROM SOLID SURFACES

(71) Applicant: ARIZONA BOARD OF REGENTS ON BEHALF OF ARIZONA STATE UNIVERSITY, Scottsdale, AZ (US)

(72) Inventor: Zhan-Gong Zhao, Tucson, AZ (US)

(21) Appl. No.: 16/041,071

(22) Filed: Jul. 20, 2018

Related U.S. Application Data
(60) Provisional application No. 62/535,693, filed on Jul. 21, 2017.

(51) Int. Cl. G01N 33/68

(52) U.S. Cl. CPC G01N 33/6851; H01J 49/0418

(57) ABSTRACT

Method and system for sequence analysis directly on a solid surface that is both high speed and high throughput are described herein, utilizing equipment available in most protein analysis facilities. For example, surface bound peptides, selectively labeled at their N-termini with a positive charge-bearing group, are subjected to controlled degradation in ammonia gas, resulting in a concatenated set of charged peptide fragments that differ by a single amino acid. The fragments are taken up in a small volume of matrix solution and analyzed by matrix assisted laser desorption/ionization (MALDI) mass spectrometry. The peptide sequences can be read directly from the resulting spectra.

### CROSS-REFERENCE TO RELATED APPLICATIONS

This application claims priority to U.S. Provisional Patent Application No. 62/535,693 filed on Jul. 21, 2017, the disclosure of which is incorporated herein by reference.

### STATEMENT REGARDING FEDERALLY SPONSORED RESEARCH OR DEVELOPMENT

This invention was made with government support under 1243082 awarded by the National Science Foundation and under HSHQDC-15-C-B0008 awarded by the Department of Homeland Security. The government has certain rights in the invention.

### BACKGROUND

Solid-phase peptide synthesis has allowed the rapid, low-cost, accurate synthesis of long peptides. Traditionally, only a small number of different peptide sequences were synthesized at once. As a result, there was not a need for high throughput quality control methods including sequence analysis. With the development of high throughput peptide synthesis and, in particular, patterned or multiplexed synthesis on solid surfaces, the number of peptides synthesized at once can easily range from thousands to millions. Methods are needed for direct sequencing of such libraries, either as a quality control step or as a means of single bead identification in combinatorial libraries. For example, on-bead screening of a “one-bead one-peptide” library is among the most popular approaches for discovery of specific peptide ligands, enzyme substrates or inhibitors. Frequently, hundreds of peptide leads are obtained from a single assay, and each lead need to be identified via sequencing. Peptide arrays on paper, glass, and silicon wafer surfaces have been used similarly for the discovery of ligands against proteins, virus particles and bacteria and for the measurement of antibody profiles used in disease diagnosis. These arrays are usually synthesized in situ and, by necessity, used without either purification or direct sequence analysis. The ability to perform sequence analysis on certain peptides in an array could both be used in lot-based quality control and to evaluate the extent of side reactions during synthesis and processing (e.g., incomplete coupling, premature alpha-amino de-protection, and side chain modifications). There is thus a need for a high throughput approach to analyze peptides in situ on solid surfaces that can be implemented with commonly available instrumentation.

Common methods of peptide sequencing at present include automated Edman degradation and Tandem mass spectrometry (MS/MS); both methods have limited throughput for various reasons. Edman degradation can be used in direct sequencing of peptides immobilized on solid resin beads, but the process is slow and requires multi-cycles of chemical transformations and HPLC analyses. Peptide sequencing using tandem MS is faster, but this method requires the peptides to be released into solution before analysis, and uses sophisticated instrumentation requiring specialized facilities and personnel. Other methods, such as ladder sequencing, have been developed for sequencing peptides in solution phase; and these methods are not applicable to direct determination of peptide sequences attached to surfaces.

### BRIEF DESCRIPTION OF DRAWINGS

The technology disclosed herein will be better understood from a reading of the following detailed description taken in conjunction with the drawings in which like reference designators are used to designate like elements, and in which:

FIG. 1. Process of mass spectroscopic analysis of peptide ladders generated in gas phase ammonia. A peptide (with amino acids AA1 through AA5 in the scheme) is synthesized on a solid support (represented as a gray circle). The N-terminal amine is labeled by a group with a permanent positive charge. This is subjected to high pressure ammonia gas which randomly cleaves peptide bonds, resulting in a series of N-terminal labeled fragments. Generally, only the charge-labeled fragments give a strong signal in the mass spectrum. Upon matrix assisted laser desorption/ionization (MALDI) mass spectrometry, one sees a series of peaks that correspond to masses separated by single amino acid molecular weights and that include the mass signature of the charge label.

FIGS. 2A-2C. Peptide degradation in gas ammonia. (A) a 5-mer peptide with its side chain protection intact was treated in gaseous ammonia. (B) the same peptide was derivatized at its N-terminus using a group containing a fixed positive charge, TMPP, and treated in gaseous ammonia. (C) the side chain protection of the charge derivatized peptide (B) were removed and treated in gaseous ammonia. The ammonia treated samples were for MALDI MS analysis.

FIGS. 3A-3C. MALDI mass spectra of peptides after treatment in gaseous ammonia. The mass spectra were not calibrated. (A) peak labeled are fragments from protected peptide, Arg(pbf)-Tyr(tBut)-Asn-Gly (SEQ ID NO:2), a: M+H, b: M+Na, c: (M-tBut)+H, d: (M−2tBut)+H, e: (M-pbf)+H. (B) the protected peptide in (A) was labeled with TMPP and the spectrum is analyzed by using Bruker Daltonics flexAnalysis 3.0; amino acid annotation and differences between peaks are determined by flexAnalysis (in red). (C) the spectrum is for the same TMPP labeled peptide in (B) after side chain deprotection, differences between adjacent peaks are annotated with amino acids using the same analytical software.

FIGS. 4A and 4B. Selective derivatization of a peptide at its N-terminus for sequence analysis. (A). an 8 mer peptide was synthesized using BOC chemistry; the peptide's side chains were deprotected by a conventional TMFSA Low-High procedure before it was derivatized with TMPP-Ac-OSu under controlled pH (8.0-8.5). (B). a few beads of the derivatized peptide were treated in ammonia gas and analyzed by MALDI mass spectrometry. The spectrum was not calibrated.

FIGS. 5A-C. MALDI mass spectra of TMPP labeled trimer peptide, TMPP-Ala-Leu-Gly-TG (TG=Tenta Gel beads). (A), the peptide was treated in ammonia gas for 23 hours at 100 Psi. (B), 5 hours at 50 Psi. (C), 2 hours at 15 PSI. The spectra were not calibrated.

FIGS. 6A-E. MALDI mass spectra of TMPP labeled six-mer peptides, TMPP-Leu-X-Ser-Gln-Asp-Gly-TG (SEQ ID NO:19). TG: tenta gel; (a) X=Lys (K); (b) X=Asn (N); (c) X=Glu (E); (d) X=Thr (T); (e) X=Trp (W).

FIGS. 7F-J. MALDI mass spectra of TMPP labeled six-mer peptides, TMPP-Leu-X-Ser-Gln-Asp-Gly-TG (SEQ ID NO:19). TG: =Tenta Gel; (f) X=His (H); (g) X=Pro (P); (h) X=Arg (R); (i) X=Try (Y); (j) X=Ile (I).

FIG. 8. Deamination of Lysine and Asparagine in ammonia gas. Deamination (−17 Da) peaks are labeled as a for Lys., b for Asn and c for Lys. No deamination is observed for the two Gln (Q) residues. The MALDI mass spectrum was not calibrated. The peptide was synthesized on Tenta Gel (TG) beads without a cleavable linker and treated in ammonia for about 1 hour at about 100 Psi.

FIG. 9. MALDI mass spectra of TMPP-labeled peptide after treatment in gaseous ammonia. The 5-peptide in supplementary FIG. 1 was labeled with TMPP using a two-step procedure. After the side chain protecting groups were removed, the labeled peptide was treated in ammonia gas before MALDI mass analysis. The mass spectrum was not calibrated. The spectrum is analyzed by using Bruker Daltonics flexAnalysis 3.0. The peptide sequence was correctly readout from the spectrum as TMPP-Arg-Tyr-Thr-Asp-Gly (SEQ ID NO:3).

FIG. 10 A-E. MALDI mass spectra of TMPP labeled six-mer peptides, TMPP-Leu-Xxx-Ser-Gln-Asp-Gly-TG (SEQ ID NO:19). TG: tenta gel; (a) Xxx=Lys (K); (b) Xxx=Asn (N); (c) Xxx=Glu (E); (d) Xxx=Thr (T); (e) Xxx=Trp (W). This figure is the same as FIGS. 4A and 4B. The display range is reset to magnify the smaller peaks. The mass spectra were not calibrated.

FIG. 11 F-J. MALDI mass spectra of TMPP labeled six-mer peptides, TMPP-Leu-Xxx-Ser-Gln-Asp-Gly-TG (SEQ ID NO:19). TG: =Tenta Gel; (f) Xxx=His (H); (g) Xxx=Pro (P); (h) Xxx=Arg (R); (i) Xxx=Try (Y); (j) Xxx=Ile (I). This figure is the same as FIG. 5 (see in main text), the display range is reset magnify the smaller peaks. The mass spectra were not calibrated.

FIG. 12. Deamination of Lysine and Asparagine in ammonia gas. Deamination (−17 Da) peaks are labeled as a for lys, b for Asn and c for Lys. No deamination is observed for the two Gln (Q) residues. This MALDI mass spectrum is part of FIG. 6 in manuscript (display range is reset to magnify the smaller peaks in FIG. 6). The spectrum was not calibrated.

### DETAILED DESCRIPTION

The technology disclosed herein is described in one or more exemplary embodiments in the following description with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present technology disclosed herein. Thus, appearances of the phrases “in one embodiment,” “in an embodiment,” and similar language throughout this specification may, but do not necessarily, all refer to the same embodiment.

The described features, structures, or characteristics of the technology disclosed herein may be combined in any suitable manner in one or more embodiments. In the following description, numerous specific details are recited to provide a thorough understanding of embodiments of the technology disclosed herein. One skilled in the relevant art will recognize, however, that the technology disclosed herein may be practiced without one or more of the specific details, or with other methods, components, materials, and so forth. In other instances, well-known structures, materials, or operations are not shown or described in detail to avoid obscuring aspects of the technology disclosed herein.

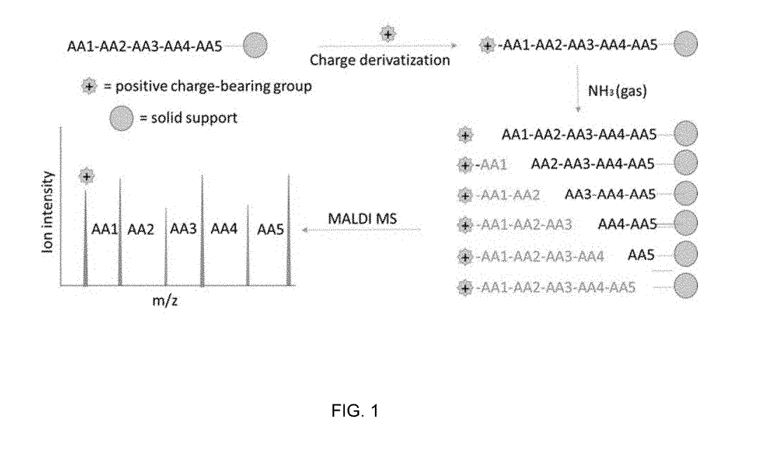

A method for peptide sequencing directly from a solid surface that is based on a one-step process involving controlled chemical fragmentation in gas phase (FIG. 1) is described herein. In certain embodiments, a peptide, attached to a solid surface such as a resin bead surface or a peptide array on a glass surface or a silicon wafer surface, is first derivatized by attaching a positive charge-bearing group at its N-terminus. In some embodiments, the derivatizing step can often be performed after the last step of in situ peptide synthesis on the solid surface before side-chain deprotection of the amino acids. In other embodiments, the derivatizing step can be performed subsequently to the deprotection of the amino acids. Furthermore, in some embodiments, the positive charge-bearing group can be N-Tris(2,4,6-trimethoxypheyl)phosphonium acetic acid (TMPP). In other embodiments, the positive charge-bearing group can be trialkylammonium acetyl (TAA). The examples provided here are not meant to be limiting and other suitable positive charge-bearing groups can also be used.

Subsequently, the derivatized peptide is subjected to controlled chemical fragmentation in ammonia gas with a pressure ranging from about 15 psi to about 100 psi. As described herein, “about” is used to show a plus or minus difference of 10% in any measurement. A family of N-terminal labeled peptide fragments is generated, each differing from the next fragment by a single amino acid. Because the cleaving step is performed in the gas phase, the cleaved fragments remain in in the same position on the surface as the peptide that gave rise to them. Cleaved fragments and the original peptides synthesized on individual beads can be extracted into a small volume (about luL) of matrix solution and analyzed with a conventional MALDI mass spectrometer or other suitable mass spectrometers. In the embodiment of peptides in an array format, matrix solution can be applied as an aerosol without substantially disturbing the position of cleaved fragments on the surface, and MALDI imaging methods or any other suitable methods can be used to perform the analysis as a function of position. The peptide sequences can be read directly from the mass spectrum. Below, sequence analysis of cleaved fragments and original peptides attached to solid resin is presented as a demonstration of one embodiment of the approach. The methods can be adapted, as suggested above, to peptide arrays.

Further, a system for sequencing a peptide directly from a solid surface is disclosed. In certain embodiments, the system comprises a container for derivatizing the in situ synthesized peptide on a solid surface; a chamber containing ammonia gas for cleaving the derivatized peptide into a set of peptide fragments, wherein the set of peptide fragments are generated by cleaving at each of the amide bonds; and a mass spectrometer for sequencing the set of peptide fragments to yield a peptide sequence of said peptide.

### EXAMPLES

Stability of Peptide Bonds in Ammonia Gas.

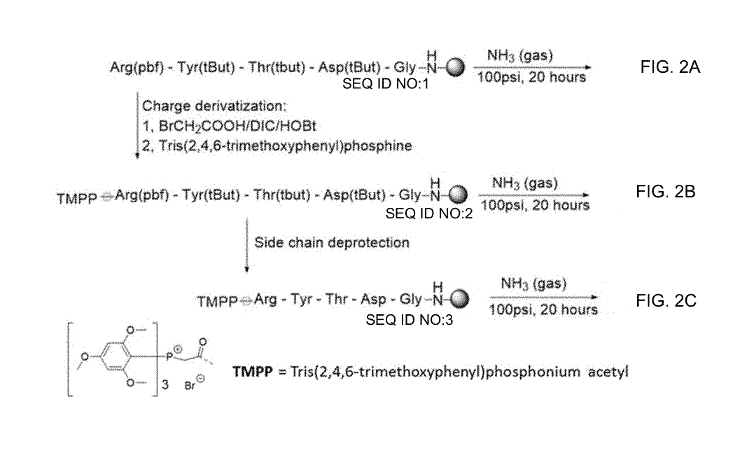

Peptide bonds can be cleaved through hydrolysis using water molecules or hydroxide ions as the nucleophile. The possibility of using high pressure ammonia gas for cleaving peptide bonds (amide bonds) was explored. Initially, a control 5-mer peptide, Arg(pbf)-Tyr(tBut)-Thr(tBut)-Asp(tBut)-Gly (SEQ ID NO:1), was synthesized on resin beads without a cleavable linker and without a permanently charge group attached to the N-terminus. For example, 2,2,4,6,7-pentamethyldihydrobenzofurane (Pbf) is a protecting group used for the protection of arginine side chains and the tert-Butyl ester is a side chain protecting group for tyrosine, threonine, and aspartate. A few beads of the peptide resin, with its side chain protection intact, were treated in ammonia gas (100 psi) for 20 hours (FIG. 2A), extracted with small volume of matrix solution and subjected to MALDI analysis.

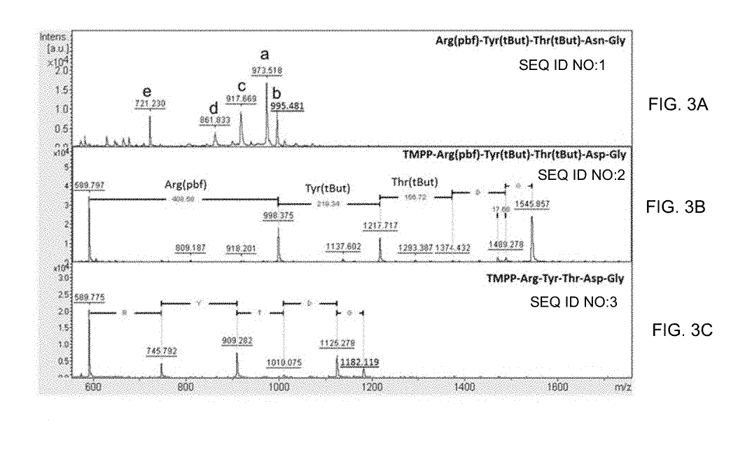

Referring to FIG. 3A for the MALDI mass spectrum, five peptide fragments were identified. Fragment (a) corresponds to the full peptide (M+H) with aspartic acid turned to asparagine during the ammonia treatment; apparently the tert-Butyl ester bond of Asp(tBut) was cleaved by ammonia (NH3) resulting in the carboxamide side chain of asparagine. Fragment (b) corresponds to the same full peptide but with a sodium adduct (M+Na). Fragments (c) and (d) are the same as fragment (a) except missing one (fragment c) or both (fragment d) tert-butyl protecting groups. Finally, fragment (e) corresponds to the full peptide as in (a) with the arginine side chain protection group, pdf, removed during ammonia treatment. There is no identifiable peak corresponding peptide bond (amide bond) breakage. However, it is demonstrated that the amide bond between the peptide and the resin bead is labile in ammonia gas, as is the pbf protection group of arginine. The loss of tert-Butyl protection is most likely due to laser fragmentation during the desorption/ionization process.

Charge Derivatization.

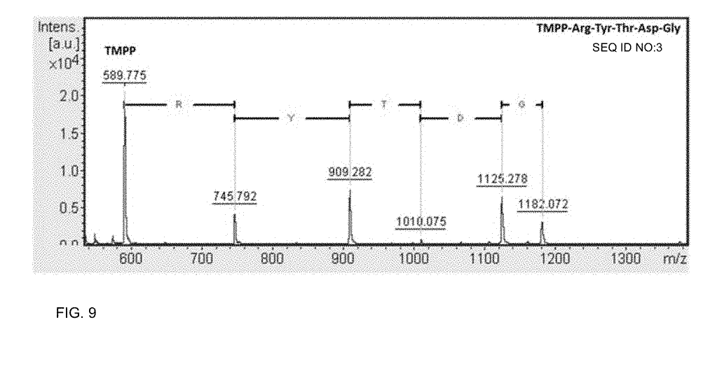

Derivatization of peptides with fixed charges has been shown to greatly increase detection sensitivity, simplify fragmentation patterns and, thus, facilitate interpretation of mass spectra in MS/MS based de novo peptide sequencing. The approach described here does not involve fragmentation in the process of mass spectra acquisition. But the inventors have observed that attaching a fixed positive charge to the N termini of the peptides (FIGS. 2A-C) indeed helped the detection of peptide fragments, possibly by increasing detection sensitivity or by facilitating peptide fragmentation in ammonia gas. Without labeling, no peptide bond breakage was observed after a short peptide was treated in high pressure of ammonia gas (FIGS. 3A-C). This indicated that peptide bonds are generally quite stable in ammonia gas and any low level of peptide bond cleavage results in fragments with too low an abundance to be detected in the mass spectrum. However, when labeled at its N-terminus with a positive charge-bearing group, the same peptide gave rise to detectable fragments from cleavages at each amide bond (peptide bond). The MALDI mass spectrum of these fragments correctly corresponded to the sequence of the peptide (FIG. 9). A phosphonium group, N-Tris(2,4,6-trimethoxypheyl)phosphonium (TMPP), was selected as the positive charge-bearing group. It has the advantage of not only being permanently charged, but it also greatly increases the molecular weight of the small peptide fragments, avoiding overlap in the spectrum with the low molecular weight ions that arise from the matrix.

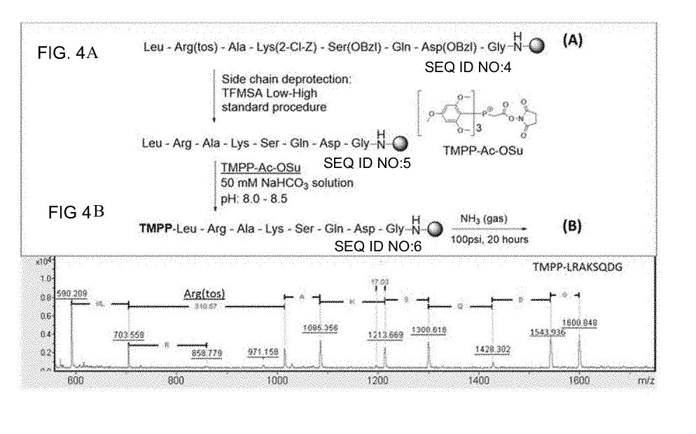

Depending on the purpose of an analysis, different approaches may be used in the labelling of a surface-immobilized peptide. The attachment of TMPP in two steps as shown in FIG. 1 is easily applied in situations where quality control of the synthesis is the primary objective. However, peptide sequencing is frequently needed for identification and characterization of unknown peptide leads selected from a ligand discovery assay (e.g., a bead library) where the side chain protection groups of peptides are previously removed. Thus a method of selectively attaching TMPP only to the N terminus of a peptide in presence of other basic residues such as Lys, His, Arg is applied. Wetzel et al. presented a selective procedure for modification of N-terminal amino groups by exploiting differences between the pKa of the N-terminal alpha amino group (˜8.0) and the pKa of the more basic amino acid side chains (˜10.5 for Lys, ˜12.0 for Arg). To test this selective derivatization approach on surface bound peptides, an 8-mer peptide that includes a lysine residue and an arginine residue was synthesized (FIG. 4A). The peptide was synthesized using BOC chemistry without a cleavable linker. After its side chains were removed by using a modified TFMSA procedure, the N-terminus of this peptide was derivatized with commercially available (Sigma) TMPP-Ac-OSu under controlled pH (0.05M NaHCO₃, pH=8.0-8.5). A few beads of this labeled peptide were then treated with ammonia gas, and a small volume (luL) of matrix solution was added to extract the peptide fragments from two of the beads for analysis by MALDI mass spectrometry. The MALDI mass spectrum (FIG. 4B) showed that only the N-terminus was derivatized as desired and gave the correct sequence of the peptide. Interestingly, the spectrum also indicated that the side chain deprotection condition is not adequate to remove side chain protecting groups of arginine and thus showed the power of this approach in detecting problems in synthesis.

Peptide Degradation in Ammonia Gas.

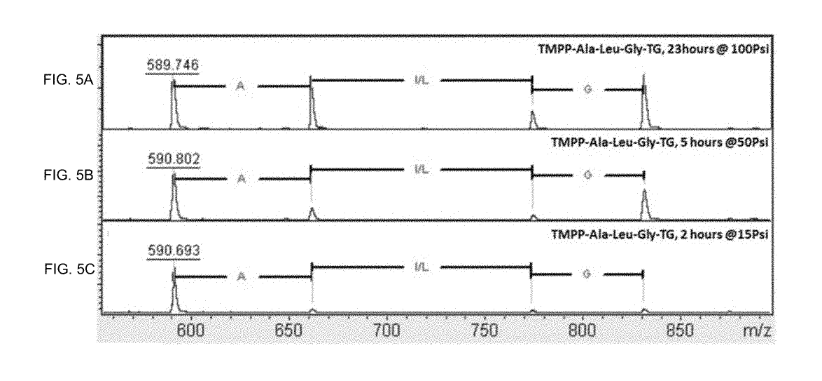

For practical application of our sequencing approach, the appropriate conditions of ammonia treatment in terms of time and pressure were determined, so that a set of peptide fragments containing peptide fragments cleaved at each amide bond (peptide bond) is generated yielding a complete peptide sequence. The inventors have observed in FIGS. 4A and 4B and also in FIGS. 3A-C that peptide bonds are quite stable, and over-degradation is unlikely to be a concern even in high ammonia pressure. This allowed the process to be performed as a function of time and pressure over wide range. A trimer peptide was synthesized on tenta Gel (TG) beads and labeled with TMPP: TMPP-Ala-Leu-Gly-TG. The peptide was then treated in ammonia gas for different periods of time and at different pressures. FIGS. 5A-5C show the MALDI mass spectra vs. time and pressure, indicating that peptide bonds are cleaved to a significant extent even with short treatment and at very low ammonia pressure (FIG. 5C). With longer time treatment at high pressure (FIG. 5A), three of the four peptide bonds were close to equal in their lability to degradation in ammonia gas. However, differences in the lability of peptide bonds to ammonia gas were observed in all three conditions in FIGS. 5a-c. Realizing that the liability of peptide bonds may be sequence dependent, the following sequences are selected as test cases: TMPP-Leu-X-Ser-Gln-Asp-Gly-TG (SEQ ID NO:19) with X=H, K, N, E, P, R, T, W, Y, I. The peptides were synthesized on Tenta Gel (TG) beads by Fmoc chemistry and without a cleavable linker. With these peptides, the effects of each amino acid, X, were determined for the cleavage of the Leu-X bond at the N-terminal side and for the cleavage of the X-Ser bond on the C-terminal side of the amino acid in question.

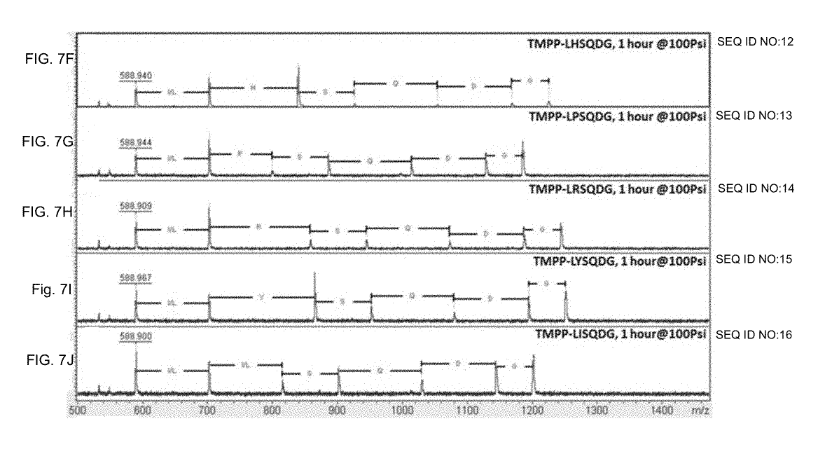

In these spectra, the intensities of molecular ions, TMPP and TMPP-Leu, are the highest and almost constant in all ten spectra, indicating that an amino acid does not affect lability of peptide bonds at its N-terminal side; the intensities of molecular ions at its C-terminal side, however, are more or less affected. For example, the intensities of molecular ions, TMPP-Leu-X, vary with the changes of amino acids and are more significant when Xxx=Asn, Thr, Trp, His, and Tyr, and relatively less significant when X=Lys, Glu, Pro, Arg, and Ile. Another interesting observation is that the intensity of molecular ion, TMPP-Leu-Thr, is relatively more significant compared to that of TMPP-Arg-Tyr-Thr as seen in FIG. 3C, indicating that the lability of a peptide bond in ammonia gas is sequence dependent. While every amino acid at the position of X is seen to have some effects on the lability of other peptide bonds at their C-terminal side, TMPP-Leu-Pro is the molecular ion worth of our particular attention: The intensity of this ion is quite low, whereas every molecular ions at its C-terminal side are relatively more significant (panel g of FIG. 7). The presence of a Proline residue may cause the peptide to adopt a turn structure that bring the TMPP moiety closer to the peptide bonds at the C-terminal side of Proline, facilitating their cleavage in ammonia gas.

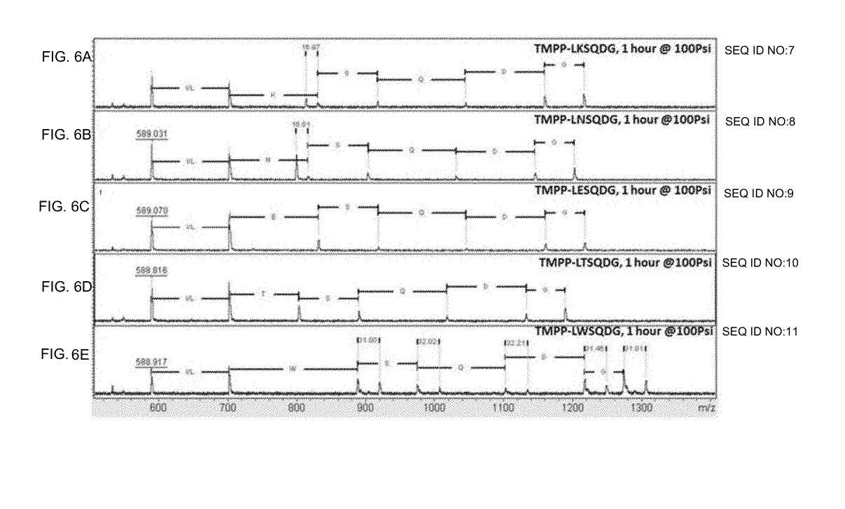

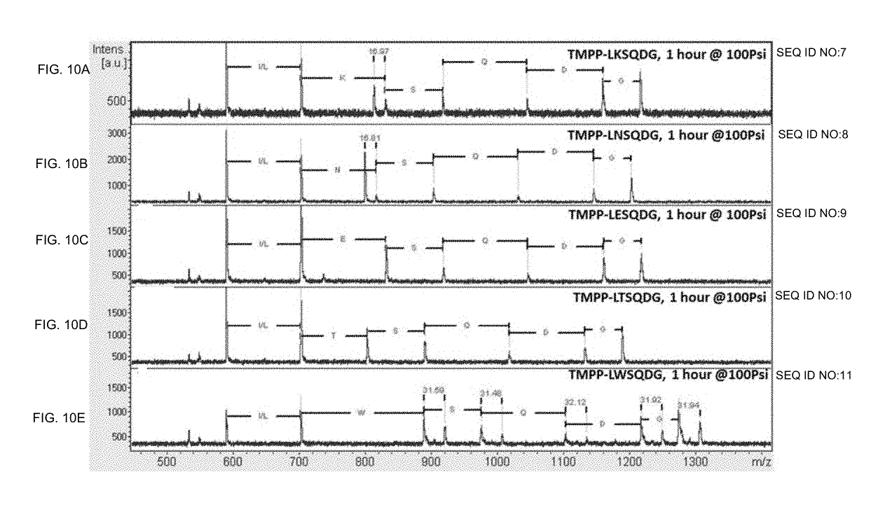

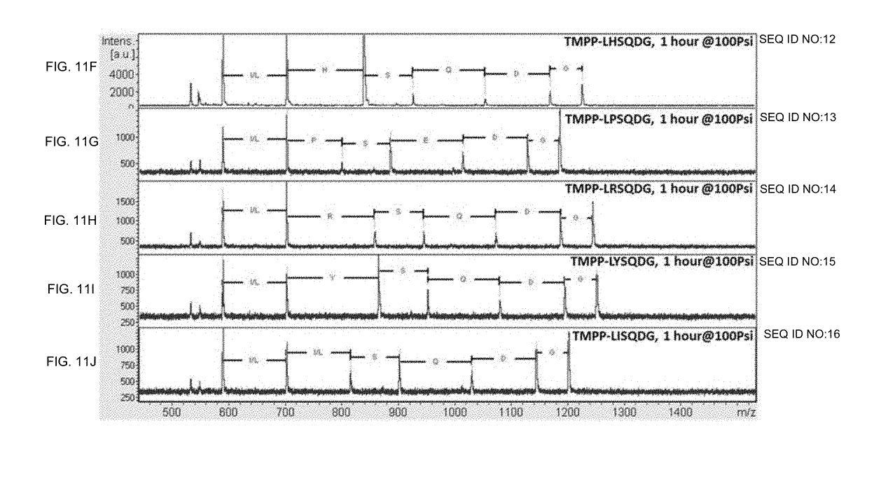

FIGS. 6 and 7 show the mass spectra of the ten sequences. The relative intensities of molecular ions, TMPP and TMPP-Leu, are the highest and almost constant in all ten spectra, indicating that an amino acid does not greatly affect lability of peptide bonds at its N-terminal side. The intensities of molecular ions due to cleavage on the C-terminal side, however, are to some extent affected. For example, the intensities of molecular ions, TMPP-Leu-X, vary with the amino acid in the X position and are greater when X=Asn, Thr, Trp, His, and Tyr, and relatively smaller when X=Lys, Glu, Pro, Arg, and Ile. In addition, the intensities of molecular ions, TMPP-Leu-X-Ser and TMPP-Leu-X-Ser-Gln (SEQ ID NO:20, also vary with the amino acid at the position of X, indicating that the effect of an amino acid on peptide bonds at its C-terminal side goes beyond one or two amino acids. For X=His, the relative intensity of TMPP-L-H is the greatest in the spectrum, while the relative intensities of TMPP-L-H-Sand TNIPP-L-H-S-Q (SEQ ID NO:21 are the lowest. To the contrary, when X=Pro, the intensity of TMPP-L-P is the lowest in the spectrum, cleavage of the next two peptides bonds results in a TMPP-tripeptide and TMPP-tetrapeptide with relatively much higher intensities. Another interesting observation is that the intensity of molecular ion, TMPP-Leu-Thr (panel d of FIG. 6), is larger than that of TMPP-Arg-Tyr-Thr as seen in FIG. 9, also indicating that the lability of a peptide bond in ammonia gas may be affected by neighboring amino acids in the peptide. At present, the effects of amino acids in a peptide on their surrounding amide bounds and the mechanism involved remain to be explored. However, cleavage at all possible positions was detectable, even using a relatively low pressure of ammonia gas over short time period, implying that the approach is robust. The axis scales in FIGS. 6 and 7 were reset to magnify the smaller peaks and presented as FIG. 10 and FIG. 11.

Amino Acid Annotation for Sequence Readout.

For sequencing purposes, the analysis primarily comprises of determining the molecular weight differences between adjacent molecular ions in the spectrum; therefore, it was not necessary to calibrate the MALDI mass spectra for accurate absolute masses. To eliminate analytical subjectivity, amino acid annotation of adjacent peaks was performed by using a software package provided by Bruker: Daltonic flexAnalysis 3.0. Using MS based sequencing methods, it is not possible to discriminate between amino acids, I and L, since their molecular weights are identical. It is also difficult to tell K and Q apart as their molecular weights differ only by 36 mDa. Depending on spectral resolution, the 1 Da difference between D and N, or E and Q, can also be challenging. However, in some cases either fortuitous or purposeful modification of amino acids can render them easier to distinguish. In FIG. 4B, there is a peak 17 Da smaller than the peak annotated to Lys. As also shown in panel a of FIG. 6, this peak most likely corresponds to the deamination of lysine. Interestingly, in all ten spectra of FIGS. 6 and 7, there is no such a peak accompanying the peak annotated as Gln in these spectra, making it possible to easily distinguish between K and Q. On the other hand, Asn does show a deamination peak and this distinguishes it easily from Asp under the conditions of this preparation (panel b of FIG. 6). Note that, in panel e of FIG. 6, there is a +32 peak found for every peptide ion that has a W residue. Most likely this peak is due to oxidation of tryptophan to N-formylkynurenine during side chain deprotection under acidic conditions.

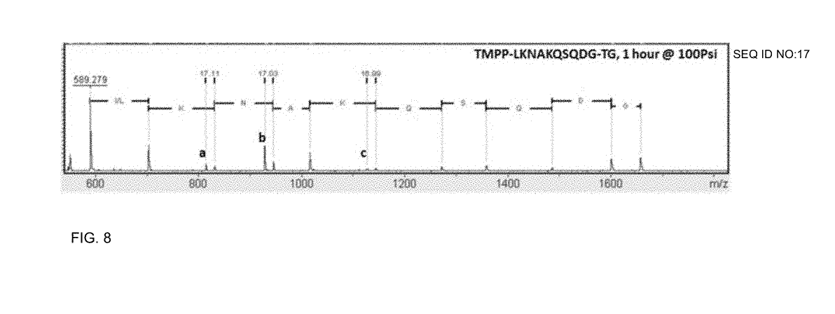

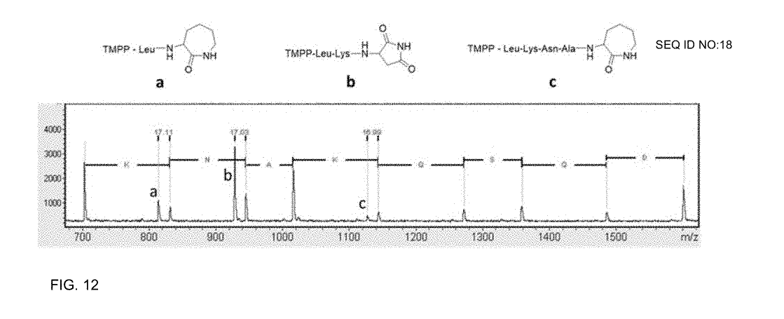

To further confirm the deamination of K and N in FIG. 6, a longer peptide, TMPP-LKNAKQSQDG (SEQ ID NO:17), was synthesized on Tenta Gel (TG) beads without a cleavable linker. With this peptide the inventors wanted to determine whether deamination of both lysine residues takes place if they are in the same peptide and also to see if both Gln (Q) residues are free from deamination as was the case in FIGS. 6 and 7. The resulting MALDI mass spectrum (FIG. 8) shows that both lysine (K) residues in the same peptide were partially deaminated, while no clear indication of deamination was observed for the two glutamine (Q) residues in the same peptide, allowing one to distinguish K and Q in this peptide. In FIG. 8, although all of the required ionized fragments are present and easily assigned, several have low intensities. FIG. 12 gives a magnified version of this spectrum, showing the weaker signals more clearly. As was observed in panel b of FIG. 6, FIG. 8 also shows significant deamination of Asn (N), thus distinguishing it from Asp (D).

Discussions

A sequence analysis approach is described that is based on a partial peptide degradation in ammonia gas. TMPP is first demonstrated to be effective as an N-terminal label to the peptides, allowing MALDI detection. The amount of cleavage of the peptides could be controlled by time exposure to the gas. The amino acid sensitivity to cleavage was determined. Finally, the inventors demonstrated that side-chain modification could be used sequence determination to distinguish between amino acids with the same or very similar molecular weights.

This current approach is both high speed and high throughput, and is particularly robust in peptide sequence analysis directly on solid surface. As the most essential step in this approach, degradation of peptides must be controlled to an appropriate extent for correct readout of the sequences; both insufficient- and over-degradation will reduce the number of fragments required for a complete sequence readout. The results show that peptide degradation in ammonia gas is easily controlled in this regard. First, unlike peptide hydrolysis in aqueous solutions, peptide (amide) bonds are relatively stable in ammonia gas so that cleavage is rare and, therefore, it is very unlikely that a peptide would be over-degraded in ammonia gas. Second, by attaching a positive charge-bearing group to the N terminus of each peptide, the resulting set of peptide fragments, even though low in abundance, are easily detectable in the MALDI mass spectra after a short period of treatment in ammonia gas (FIGS. 5A-5C). Depending on the specific needs of the analysis, the peptide can be derivatized at its N-terminus either by a two-step process before side chain deprotection, or by a pH controlled one-step process after the peptide is deprotected.

As with other MS based sequencing approaches, unambiguously distinguishing between certain amino acids with identical or similar molecular weights (I/L, K/Q, Q/E, and N/D) presents a challenge, particularly if one hopes to use the method with lower resolution, less expensive, more available mass spectrometers. This problem can be alleviated at least for K/Q and N/D by considering fortuitous or purposeful modifications of these amino acids, as described above. It is not clear what the mechanism is for deamination of K and N in this process nor is it clear why N gives a deamination product while Q does not. However, the ability to discriminate between K and Q, as well as N and D, without having to perform specific modification chemistry is a useful aspect of this approach.

In summary, the fact that amide bond cleavage in this approach is carried out by gas phase ammonia, without addition of other chemicals to the sample, minimizes the manipulations involved and makes this approach ideal for high speed and high throughput sequence analysis. No cleavable linker is required during synthesis as the peptides are not released, only fragmented in situ, for analysis. The positive charge-bearing group added to the N-terminus provides the sensitivity required to detect low abundance fragments needed in the sequencing analysis and serves as an internal reference in MALDI MS analysis. After a small volume of matrix solution is applied, MALDI mass spectra can be acquired with an inexpensive, widely available desktop mass spectrometer. The peptide sequence can be read directly from the mass spectra without the need of sophisticated processing. Finally, because the peptide fragments remain where the peptide is synthesized during the process, this approach is particularly suited for multiplex analysis of peptide sequences in microarray format, an important task that is difficult to perform using other approaches.

Methods

Solid Phase Peptide Synthesis.

Tenta Gel amino resin (EMD Millipore, Billerica, Mass.) was used in solid phase synthesis of all peptides reported here. The stepwise assembly of the peptide sequence was performed via addition of the corresponding Fmoc- or Boc-protected amino acids in presence of Diisopropylcarbodiimide (DIC) and 6-chloro-1-hydroxytriazole. Both Fmoc- and BOC-protected amino acids were from AAPPTEC (Louisville, Ky.). For peptide synthesis with Fmoc-protected amino acids, Fmoc was removed by treatment of peptide resin in 20% piperidine in DMF (5 min+15 min); after the synthesis, the side chain protection were removed by treatment of peptide resin in a solution of trifluoroacetic acid (TFA, 90%), water (2.5%), Ethanedithiol (2.5%), and triisopropylsilane (5%) for 3 hours. The resin was then washed with dichloromethane and Methanol, dried before use. In Boc-based synthesis, the BOC group was removed in 50% trifluoroacetic acid in dichloromethane (30 min); the side chain protections of the peptide were removed by following a “low-high trifluoromethanesulfonic acid (TFMSA)” method: the dried resin (50 mg) was first suspended in a low cleavage solution of m-Cresol (50 uL), Dimethyl sulfide (150 uL), trifluoroacetic acid (250 uL), Trifluoromethanesulfonic acid (TFMSA, 50 uL), and 3,6-Dioxa-1,8-octane-dithiol (10 uL) and shaken for 3 hours at 0-5° C.; the resin, after the low cleavage solution was removed, was suspended in a high cleavage solution of trifluoroacetic acid (500 uL), thioanisole (50 uL), TFMSA (50 uL), and 3,6-Dioxa-1,8-octane-dithiol (15 uL) and shaken for 1.5 hours at room temperature. The resin was then washed with Dichloromethane, Methanol, and dried before use.

Tmpp Derivatization.

Peptides can be derivatized with TMPP (tris(2,4,6-trimethoxyphenyl)phosphonium acetic acid) at their N-terminal either during the synthesis for quality analysis or after the synthesis is complete and side-chain deprotected for lead identification. During the synthesis, TMPP can be attached to the peptides at their N-terminal in two steps as shown in FIGS. 2A-C; typically, a small sample or resin (˜1-5 mg) is removed from the synthesis and washed with Methanol/Dichloromethane in a small column and suspended in a solution of bromoacetic acid (0.1M), 6-chloro-1-hydroxybenzotriazole (0.1M), and Diisopropylcarbodiimide (0.1M) in N-methylpyrrolidinone (0.2 mL) and shaken for 1 hour at room temperature. The resin is then washed with N-methylpyrrolidinone to completely remove unreacted chemicals before suspended in a solution of tris(2,4,6-trimethoxyphenyl)phosphine (0.1M) in N-methylpyrrolidinone; the reaction is allowed to proceed in room temperature for 2 hours to ensure the Bromine atom is completely displaced by the phosphine, forming a positive charge-bearing phosphonium at the N terminus of a peptide. The derivatized resin is ready for ammonia gas degradation with/without side chain deprotection (see FIGS. 3A-C).

For selective derivatization of a fully deprotected peptide at its N-terminus in presence of other basic amino acids such as Lysine and Arginine, a small sample of peptide on Tenta Gel beads in a column (sample size varied from a few hundred beads to a few milligrams depending on experimental purpose) are suspended and shaken in aqueous solution of NaHCO₃ (100 mM, pH=8.0-8.5) for 30 min; after the buffer solution is removed, a solution of TMPP-Ac-Suc (1 mg) in 500 uL acetonitrile-100 mM NaHCO₃ (1:9) is added to the resin sample and shaken for 1 hour at room temperature. The resin sample is then washed with water (5×1 min), acetonitrile (3×1 min), Methanol (3×1 min) and DCM (3×1 min). The resin is then dried in vacuo before subjected to ammonia treatment.

Peptide degradation in ammonia gas.

A stainless steel gas chamber was designed to hold sample vials of various sizes; a stainless steel cap, equipped with inlet and outlet connectors, was screw-fastened to the top of the chamber to withstand pressures well over 100 psi. An ammonia tank was linked to the chamber through a polypropylene inlet tubing and a gas pressure gauge, through which the pressure in the chamber can be adjusted and calibrated; a valve was placed at the outlet and linked to a water trap to absorb ammonia gas coming out of the chamber. The degradation process started by flowing ammonia gas through the system; while ammonia was completely captured in the water trap, air coming out of the chamber will bubble through the water trap until no bubble out. Ammonia pressure was then increased to desired level after closing the valve on the outlet and allow the reaction to proceed undisturbed for a desired period of time. After the reaction, resin beads in vials were allowed to vent out residual ammonia before MS analysis.

MS Analysis.

MALDI mass spectra were all recorded on Bruker Microflex™ spectrometer, which is an entry-level instrument ideal for non-expert user without specialized training. The spectrometer is equipped with a nitrogen laser (337 nm, 3 ns pulse width, 150 μJ pulse energy) with variable repetition rate, and a microScout ion source with state-of the art pulsed ion extraction. flexControl™ software module was used for instrument control, flexAnalysis™ were used for all data analysis. As matrix, a saturated solution of α-cyano-4-hydroxycinnamic acid (CHCA) was made in 50% aqueous acetonitrile containing 0.1% trifluoroacetic acid (TFA). For analysis of individual beads, the selected beads were first suspended in minimum volume of acetonitrile and placed in a small petri dish to spread the beads apart and let the solvents evaporate. A small volume (1-2 μL) of matrix solution was then dispensed through a micropipette tip onto a bead; the solution, after a few seconds, was retrieved to the tip and spotted onto the MALDI target plate for analysis. For analysis during synthesis, as described above, a small sample (1-5 mg) was removed from synthesis, placed in a small column with frit, labeled with TMPP at the N-terminus, and deprotected. After gaseous degradation, the resin was suspended in minimum volume of 50% aqueous acetonitrile to extract the degraded peptides; the extract (1 μL) is then added to 10 μL matrix solution, and 1 μL of the mixture was then placed on the MALDI target plate for analysis.

While the preferred embodiments of the present technology have been illustrated in detail, it should be apparent that modifications and adaptations to those embodiments may occur to one skilled in the art without departing from the scope of the present technology.

### REFERENCES

1. Merrifield, R. B., Solid Phase Peptide Synthesis. I. The Synthesis of a Tetrapeptide. Journal of the American Chemical Society, 1963. 85(14): p. 2149-2154.
2. Lam, K. S., et al., A new type of synthetic peptide library for identifying ligand-binding activity. Nature, 1991. 354(6348): p. 82-84.
3. Meldal, M., et al., Portion-mixing peptide libraries of quenched fluorogenic substrates for complete subsite mapping of endoprotease specificity. Proceedings of the National Academy of Sciences of the United States of America, 1994. 91(8): p. 3314-3318.
4. Wu, J., Q. N. Ma, and K. S. Lam, Identifying Substrate Motifs of Protein Kinases by a Random Library Approach. Biochemistry, 1994. 33(49): p. 14825-14833.
5. I, S., et al., Identification of a Peptide Inhibitor against Glycosomal Phosphoglycerate Kinase of Trypanosoma-Brucei by a Synthetic Peptide Library Approach. Bioorganic & Medicinal Chemistry, 1995. 3(3): p. 257-265.
6. Meldal, M. and I. Svendsen, Direct Visualization of Enzyme-Inhibitors Using a Portion Mixing Inhibitor Library Containing a Quenched Fluorogenic Peptide Substrate 0.1. Inhibitors for Subtilisin Carlsberg. Journal of the Chemical Society-Perkin Transactions 1, 1995(12): p. 1591-1596.
7. Frank, R., Spot-synthesis: an easy technique for the positionally addressable, parallel chemical synthesis on a membrane support. Tetrahedron, 1992. 48(42): p. 9217-9232.
8. Fodor, S. P., et al., Light-directed, spatially addressable parallel chemical synthesis. Science, 1991. 251(4995): p. 767-773.
9. Legutki, J. B., et al., Scalable high-density peptide arrays for comprehensive health monitoring. Nature Communications, 2014. 5.
10. Nidhi Gupta, J. L., Valeriy Domenyuk, Zhan-Gong Zhao, Stephen Albert Johnston, Chris W. Diehnelt, Whole Virus Screening to Develop Synbodies for Influenza Virus. Submitted, 2016.
11. Domenyuk, V., et al., A Technology for Developing Synbodies with Antibacterial Activity. Plos One, 2013. 8(1).
12. Legutki, J. B., et al., A general method for characterization of humoral immunity induced by a vaccine or infection. Vaccine, 2010. 28(28): p. 4529-4537.
13. Sykes, K. F., J. B. Legutki, and P. Stafford, Immunosignaturing: a critical review. Trends in Biotechnology, 2013. 31(1): p. 45-51.
14. Stafford, P., et al., Immunosignature system for diagnosis of cancer. Proceedings of the National Academy of Sciences of the United States of America, 2014. 111(30): p. E3072-E3080.
15. Restrepo, L., et al., Application of Immunosignatures to the Assessment of Alzheimer's Disease. Annals of Neurology, 2011. 70(2): p. 286-295.
16. Edman, P. and G. Begg, A Protein Sequenator. European Journal of Biochemistry, 1967. 1(1): p. 80-91.
17. Matsueda, G. R., E. Haber, and M. N. Margolies, Quantitative Solid-Phase Edman Degradation for Evaluation of Extended Solid-Phase Peptide-Synthesis. Biochemistry, 1981. 20(9): p. 2571-2580.
18. Angel, T. E., et al., Mass spectrometry-based proteomics: existing capabilities and future directions. Chemical Society Reviews, 2012. 41(10): p. 3912-3928.
19. Steen, H. and M. Mann, The abc's (and xyz's) of peptide sequencing. Nat Rev Mol Cell Biol, 2004. 5(9): p. 699-711.
20. Frank, A. M., Predicting Intensity Ranks of Peptide Fragment Ions. Journal of Proteome Research, 2009. 8(5): p. 2226-2240.
21. Chait, B., et al., Protein ladder sequencing. Science, 1993. 262(5130): p. 89-92.
22. Gu, Q.-M. and G. D. Prestwich, Efficient peptide ladder sequencing by MALDI-TOF mass spectrometry using allyl isothiocyanate. The Journal of Peptide Research, 1997. 49(6): p. 484-491.
23. Zhong, H., et al., Protein sequencing by mass analysis of polypeptide ladders after controlled protein hydrolysis. Nat Biotech, 2004. 22(10): p. 1291-1296.
24. Shen, T.-L., et al., Evaluation of charge derivatization of a proteolytic protein digest for improved mass spectrometric analysis: de novo sequencing by matrix-assisted laser desorption/ionization post-source decay mass spectrometry. Journal of Mass Spectrometry, 1999. 34(11): p. 1154-1165.
25. Roth, K. D. W., et al., Charge derivatization of peptides for analysis by mass spectrometry. Mass Spectrometry Reviews, 1998. 17(4): p. 255-274.
26. Wetzel, R., et al., A general method for highly selective crosslinking of unprotected polypeptides via pH-controlled modification of N-terminal .alpha.-amino groups. Bioconjugate Chemistry, 1990. 1(2): p. 114-122.
27. Kuyama, H., et al., A method for N-terminal de novo sequence analysis of proteins by matrix-assisted laser desorption/ionization mass spectrometry. Anal Biochem, 2008. 380(2): p. 291-6.
28. Spengler, B., et al., Peptide sequencing of charged derivatives by postsource decay MALDI mass spectrometry. International Journal of Mass Spectrometry and Ion Processes, 1997. 169: p. 127-140.
29. Yajima, H., et al., Trifluoromethanesulphonic acid, as a deprotecting reagent in peptide chemistry. Journal of the Chemical Society, Chemical Communications, 1974(3): p. 107-108.
30. Pan, B., M. S. Ricci, and B. L. Trout, Molecular Mechanism of Acid-Catalyzed Hydrolysis of Peptide Bonds Using a Model Compound. The Journal of Physical Chemistry B, 2010. 114(13): p. 4389-4399.
31. Smith, R. M. and D. E. Hansen, The pH-Rate Profile for the Hydrolysis of a Peptide Bond. Journal of the American Chemical Society, 1998. 120(35): p. 8910-8913.
32. Fountoulakis, M. and H.-W. Lahm, Hydrolysis and amino acid composition analysis of proteins. Journal of Chromatography A, 1998. 826(2): p. 109-134.

What is claimed is:

1. A method for sequencing a peptide, comprising:
derivatizing the peptide containing N peptide bonds;
cleaving the derivatized peptide into a set of peptide fragments, wherein the set of peptide fragments are generated by cleaving at each of the N peptide bonds;
sequencing the set of peptide fragments to yield a peptide sequence of said peptide.

2. The method of claim 1, wherein the peptide is coupled to a solid surface.

3. The method of claim 2, wherein the peptide is synthesized in situ on the solid surface.

4. The method of claim 2, wherein the peptide is obtained from protein digestion and attached to the solid surface.

5. The method of claim 2, wherein the solid surface is a resin bead.

6. The method of claim 2, wherein the solid surface is a peptide microarray, wherein the microarray comprises a glass or on a silicon wafer.

7. The method of claim 1, wherein the set of peptide fragments comprises various lengths.

8. The method of claim 1, wherein the derivatizing step comprises attaching a positive charge-bearing group to a N-terminus of a peptide.

9. The method of claim 8, wherein the positive charge-bearing group is N-Tris(2,4,6-trimethoxypheyl)phosphonium acetic acid (TMPP).

10. The method of claim 8, wherein the positive charge-bearing group is trialkylammonium acetyl (TAA).

11. The method of claim 8, wherein the attaching a positive charge-bearing group to a N-terminus of a peptide step is performed prior to a deprotection step of the peptide.

12. The method of claim 8, wherein the attaching a positive charge-bearing group to a N-terminus of a peptide step is performed subsequently to the deprotection of a the peptide.

13. The method of claim 11 or 12, wherein the deprotection step comprises removing a protecting group from an amino acid.

14. The method of claim 1, wherein cleaving step comprises treating the peptide in ammonia gas for a length of time.

15. The method of claim 14, wherein the ammonia gas has a pressure ranging from about 15 psi to about 100 psi.

16. The method of claim 14, wherein the length of time is about 1 hour to about 5 hours.

17. The method of claim 1, wherein the sequencing step further comprising extracting the set of peptide fragments by a matrix solution and analyzing the set of peptide fragments by a mass spectrometry.

18. The method of claim 17, wherein the mass spectrometry is a matrix assisted laser desorption/ionization (MALDI) mass spectrometry.

19. A system for sequencing a peptide directly from a solid surface, comprising:
a container for derivatizing the peptide containing N peptide bonds;
a chamber containing ammonia gas for cleaving the derivatized peptide into a set of peptide fragments, wherein the set of peptide fragments are generated by cleaving at each of the N peptide bonds; and
a mass spectrometer for sequencing the set of peptide fragments to yield a peptide sequence of said peptide.

20. The system of claim 19, wherein the solid surface is a resin bead or a peptide microarray.
